(19) United States
(12) Patent Application Publication
(10) Pub. No.: US 2018/0303590 A1
(43) Pub. Date: Oct. 25, 2018

(54) Freehand Ring and Method of Use Thereof

(71) Applicants: Alisha Love Luman, Scottsdale, AZ (US); Jeremy Luman, Scottsdale, AZ (US)

(72) Inventors: Alisha Love Luman, Scottsdale, AZ (US); Jeremy Luman, Scottsdale, AZ (US)

(21) Appl. No.: 15/959,084

(22) Filed: Apr. 20, 2018

Related U.S. Application Data
(60) Provisional application No. 62/488,682, filed on Apr. 21, 2017.

(51) Int. Cl. A61C 19/00; A61L 2/18

(52) U.S. Cl. CPC A61C 19/006; A61C 3/00; A61L 2/18; A61C 19/002

(57) ABSTRACT

A finger mounted device for assisting a dental procedure mainly comprises: a ring gripping a finger and a platform provided with an upstanding sidewall, two bosses and at least one recessed compartment, wherein the ring and the platform are formed in a continuous structure. Further, the device is modular as the platform can engage and disengage with different types of inserts such as a form insert and a deep well insert, wherein both inserts are removably attached atop of the platform. The form insert is disposable and replaceable and serves a cleaning surface to wipe a dental tool clean. The deep well insert enables the device to hold more volume of a dental substance.

### RELATED APPLICATION

This application claims priority from U.S. provisional patent application No. 62/488,682 filed Apr. 21, 2017, and hereby incorporates subject matters of the provisional application in its entirety.

### FIELD OF THE INVENTION

The present invention relates generally to the field of dental accessories and more specifically to a finger-worn dental tool.

### BACKGROUND

In the practice of dentistry, most dental tools, especially designed to scrap or clean teeth or gum lines, are inevitably contaminated or blocked by debris such as food, plaque, tartar, blood, gum tissue, other build up materials near the gum line, etc. Such debris collected on the tips of the dental tools often interfere the dental procedure as the tips are no longer able to pick up debris or obstruct the site of the dental practitioner. Hence, the dental practitioner frequently needs to clean the contaminated tips in the middle of a dental procedure.

To clean the tips of dental tools, typically, the dental practitioner pauses the procedure, takes hands out of the patient's mouth, finds a gauze pad or other cloth, and wipes the tips clean. Such cleaning action most likely requires both hands, thereby stops the ongoing procedure and happens frequently as the tips of dental tools are constantly contaminated by touching the gum line or teeth during the dental procedure. Such repetitive cleaning action using both hands could interrupt the dental procedure and even pose a risk of injury to the practitioner and the patient.

In similar consideration of procedural interruption and injury risks, not only dental cleaning but also dental polishing procedures need a freehand device assisting the dental practitioner. Many dental practitioners work alone with their patients especially during polishing of dental surfaces. Whenever the practitioner needs to apply a dental substance to an oral cavity such as liquid, gel, paste, etc., it is quite cumbersome and inefficient for the practitioner to hold the substance in one hand while applying it with another hand to patient's teeth and even keeping the patient's tongue and oral tissue away from the working area. Therefore, such action requires both hands as well as attention on multiple working sites, whereby distracts the practitioner and consumes valuable procedural time. Plus, those inefficient steps repetitively occur during a dental polishing procedure.

Accordingly, there is a need for a suitable solution adding efficiency on dental procedures that is multifunctional, modular, portable, self-contained, unitary, handheld, disposable, reusable and cost efficient. There are various attempts proposing handheld dental accessories enhancing efficiency of dental procedures: U.S. Pat. No. 8,584,298 to Viscomi; U.S. Pat. No. 9,474,584 to Oien; U.S. Pat. No. 6,257,888 to Barham; U.S. Pat. No. 7,014,463 to Savoia; U.S. Pat. No. 4,844,308 to Porteous; and U.S. Pat. Pub. No. 2009/0126134 to Whipple.

U.S. Pat. No. 6,257,888 to Barham discloses hand and finger supported dental accessories. The invention can be handheld in two ways: adhesively anchored on the upper side of one hand or resiliently gripped on a finger. Considering most dental practitioners' wear latex or vinyl gloves, the adhesiveness likely fails to secure the invention on the hand as the gloves contract, stretch or distort during a dental procedure. Further, the invention has a tray-like holder containing an absorbent medium for cleaning dental tools and a well for receiving a pre-formed vial of polishing paste. Hence, the well is shaped to hold the vial of polishing paste, so the fixed interior volume of the well limits how much the dental substance can be taken. Likewise, the absorbent medium is pre-secured in the tray-like holder, neither removable nor replaceable. Therefore, the invention is not modular and limited to single-use.

U.S. Pat. No. 4,844,308 to Porteous discloses a finger mounted dental cup to hold dental paste and cleaning agent. Although the invention has a ring-like finger mount and a space in the form of a cup to hold dental paste and cleaning agent, it does not provide a cleaning function of a dental tool. Plus, the cup is not mounted atop of the ring-like structure. Instead, the cup is mounted to the side of the ring-like structure. Thereby, the cup may likely push the adjacent finger and interfere its movement during a dental procedure.

U.S. Pat. No. 8,584,298 to Viscomi discloses a finger mounted instrument that comprises a finger guard and a disposable form attached thereto. The form is used for cleaning a dental instrument and further impregnated with a medicament for disinfecting the dental instrument during a dental operation. However, it does not provide a separate space to hold additional dental substances (e.g., polishing paste).

U.S. Pat. Pub. No. 2009/0126134 to Whipple discloses a ring-like receptacle where a grooved or cut sponge is positioned, wherein the sponge is impregnated with an antibacterial solution and used for cleaning a dental tool. The invention is limited to use for cleaning a dental tool, not to serve multiple functions. In addition, the cleaning occurs at one place (i.e., the sponge) and does not provide a separate space to hold additional substances (e.g., polishing paste).

U.S. Pat. No. 7,014,463 to Savoia discloses a dental hygiene accessory for cleaning a dental instrument. It has a ring-like retainer, a disk-like platform, bristles upstanding from a central area thereof, and an annular body of a soft dental debris surrounding the bristles. The disk-like platform is supported by the retainer enclosing a finger. The bristles and the soft dental debris are used for cleaning dental tools. However, the invention is limited to use for cleaning a dental tool and not reusable.

U.S. Pat. No. 9,474,584 to Oien discloses a dental instrument servicing system provided with a holder and a cushion. The invention serves multipurpose such as receiving, retaining, storing, and cleaning various dental instruments during a dental procedure. However, it is different from the present invention since the dental instrument servicing system is not supported by a finger and has a substantially different configuration.

None of the aforementioned attempts, taken either singly or in combination, do not suggest the present invention as claimed in this patent application. Therefore, there is a need to have a handheld device for cleaning dental tools and receiving and holding dental substances, which is disposable, adjustable, reusable, modular, compact, self-contained, cost efficient, and portable, while efficiently performing multiple and necessary functions.

### SUMMARY

For improving issues aforementioned, the present invention (herein, the present invention and the freehand ring are interchangeable) discloses a finger mounted device for multifunctional use during a dental procedure. The present invention mainly comprises: a discontinuous ring 110 gripping a finger and a platform 140 provided with an upstanding sidewall 146 and bosses 148A/B and at least one recessed compartment 150A/B, wherein the ring 110 and the platform 140 are formed in a continuous structure. The continuous structure may be reusable after sterilization. Further, the device is modular as the platform 140 may engage and disengage with different types of inserts such as a form insert 200 and a deep well insert 300. Depending on the use, either the form insert 200 or the deep well insert 300 is removably attached atop of the platform 140. The form insert 200 is disposable and replaceable and serves a cleaning surface to wipe a dental tool. The deep well insert 300 enables the device to hold more volume of gel or liquid.

These and other embodiments are described further below with reference to the figures.

### BRIEF DESCRIPTION OF THE DRAWINGS

To facilitate understanding, the same reference numerals have been used, where possible, to designate common components presented in the figures. The drawings are depicted schematically and not to scale. Various embodiments can readily be understood by considering the following detailed description in conjunction with the accompanying drawings.

FIG. 1 is a perspective view of an embodiment of the device 100 showing the ring 110 and a front surface 142 of the platform 140 having two recessed compartments 150A/B.

FIG. 2 is a perspective view of the embodiment represented in FIG. 1, showing the ring 110 and a back surface 144 of the platform 140.

FIG. 3 is a top view of the embodiment represented in FIG. 1, showing the front surface 142 of the platform 140.

FIG. 4 is a perspective view of another embodiment of the device 100/200, showing a form insert 200 engaging with the embodiment represented in FIG. 1.

FIG. 5 is a perspective view of yet another embodiment of the device 100/300, showing a deep well insert 300 engaging with the embodiment represented in FIG. 1.

### DETAILED DESCRIPTION

A detailed description of various embodiments is provided below along with accompanying figures. The detailed description is provided in conjunction with such embodiments, but is not limited to such examples. It is envisioned that a person skilled in the art will appreciate other possible configurations of the present invention, and that any such work around will fall under the scope of the present invention.

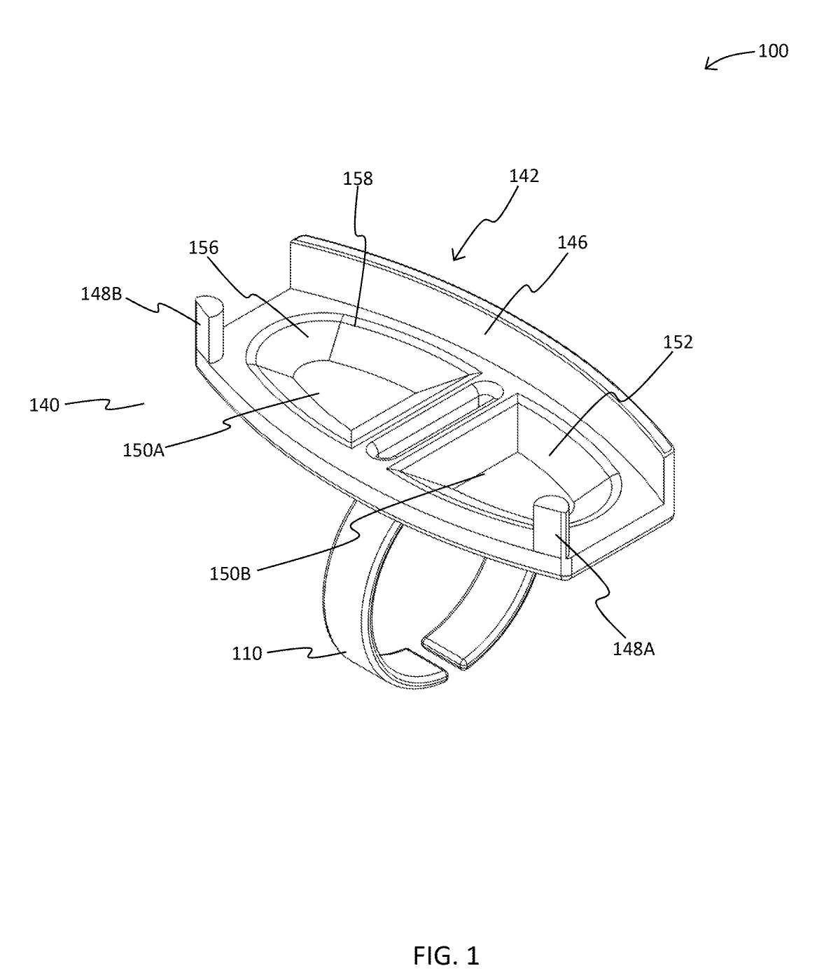

Referring to nonexclusive examples, FIGS. 1 to 5, the present invention describes the device 100/200/300 for assisting a dental procedure. The present invention mainly comprises a ring 110 and a platform 140, and further a form insert 200 and a deep well insert 300.

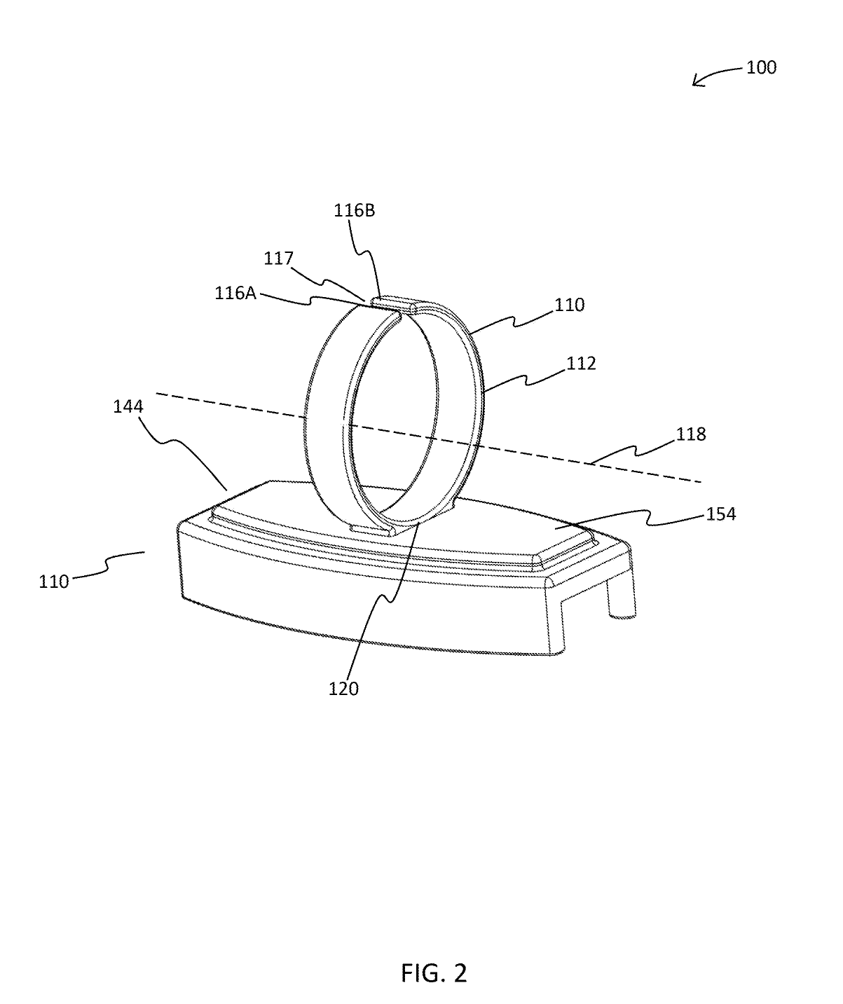

FIGS. 1, 2 and 3 represent one embodiment comprising a ring 110 and a platform 140. Referring to FIG. 2, the ring 110 comprises a substantially circular band 112, a finger passage axis 118, a band top 120, and a pair of distal ends of the band 116A/B, wherein the band ends 116A/B are positioned proximate each other but spaced apart so form a gap 117 therebetween to be adjustable for accommodating fingers of various sizes. The band 112 encloses and grips a finger and supports the platform 140 atop of the finger.

Referring to FIG. 1, the platform 140 has a generally rectangular tray-like configuration, and comprises a front surface 142, a back surface 144, a long axis 160, and short axis 162. The front surface 142 is further provided with an upstanding sidewall 146 extending from a long periphery of the platform 140, two upstanding bosses 148A/B extending from two vertexes opposite of the upstanding sidewall 146, and at least one compartment 150A/B (hereby, this embodiment has two compartments) defined by an opening 152 at the front surface 142, a base 154 recessed from the front surface 142, and an inner wall 156 extending upwardly from an outer edge of the base 154 in order to form an interior space for receiving or holding gel or liquid like dental substances. The back surface 144 is opposed to the front surface 142. The band top 120 of the ring 110 is integrally connected at the center of the back surface 144 so as to mechanically support the platform 140 as shown in FIG. 2.

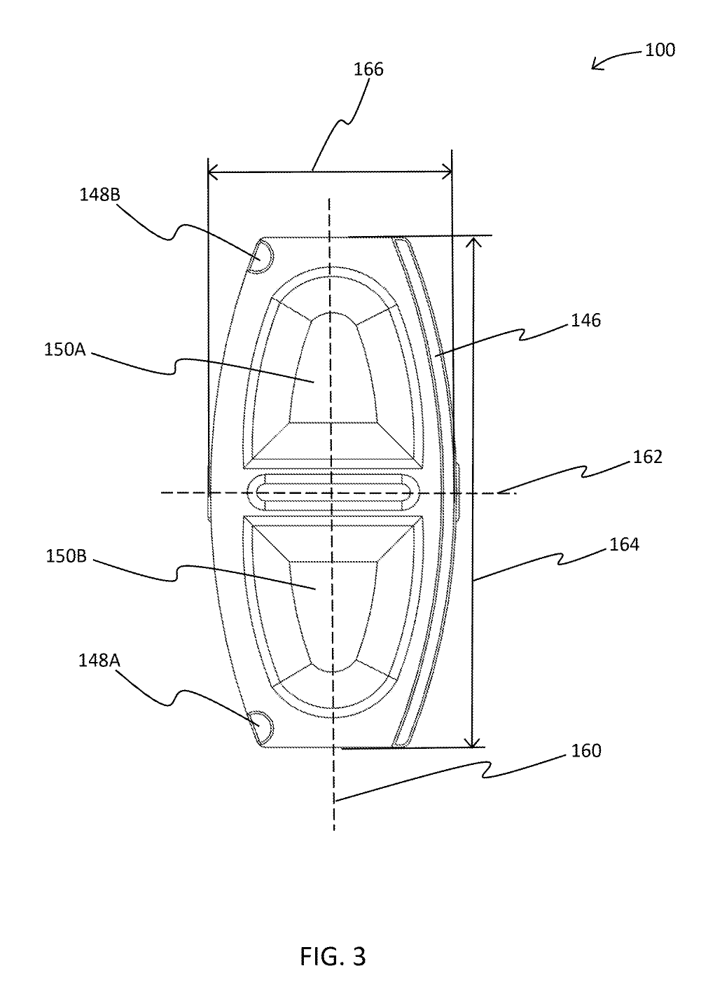

FIG. 3 represents a top view of the embodiment represented in FIG. 1. Overall, the platform 140 has a general rectangular shape. Two long sides and two short sides thereof are considered length 164 and width 166, respectively. The long axis 160 of the platform 140 crosses the center of the width 166, and the short axis 162 of the platform 140 crosses the center of the length 164. Referring to FIGS. 2 and 3, the finger passage axis 118 is generally parallel to the long axis 160 but perpendicular to the short axis 162. In the other word, the length 164 of the platform 140 is generally in the direction of the length of the finger.

Further, the continuous construction of the ring 110 and the platform 140 may comprise compliant resin materials such as Dupont Zytel FG101L NC010, Dupont Zytel FG151L NC010, or Dupont Crastin FGS600F040 NC010. Therein, chemical, chill, or heat sterilization may be applicable to the device 100, so the device 100 is reusable. Such monolithic construction may be smoothed with general curves for aesthetic and comfort reasons. The device 100 in FIGS. 1, 2 and 3 may be used as it is without other modular attachments (i.e., the form insert 200 or the deep well insert 300). For example, the platform 140 may hold a preformed vial of polishing paste or be provided with prefilled polishing paste in the compartment 150A/150B. Alternatively, the platform 140 may comprise multiple recessed compartments 150A/150B that are prefilled with different types of substances (e.g., grit and non-grit type polishing paste) and sealed with a peelable foil cover.

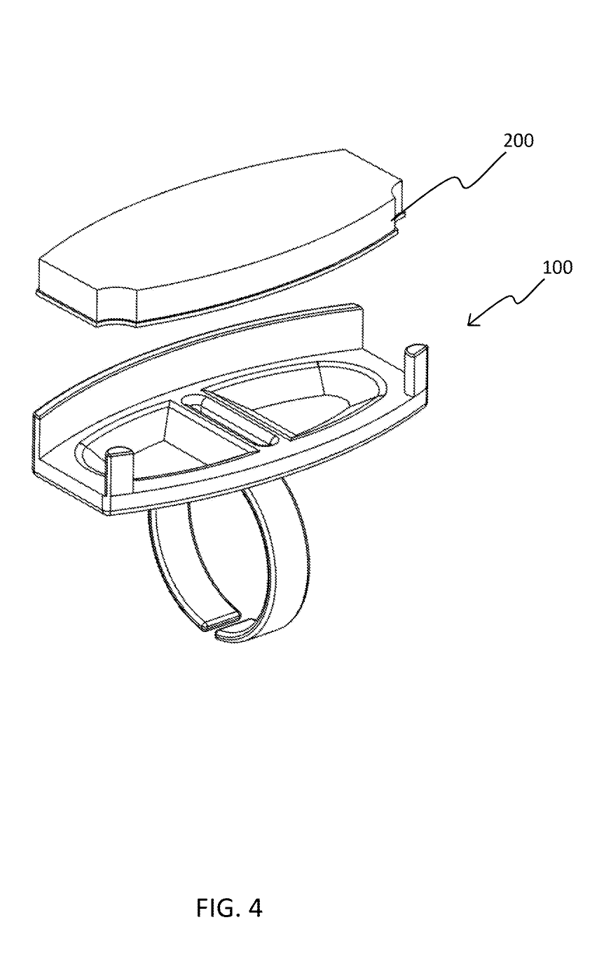

In another embodiment in FIG. 4, the device 100 is further provided with a form insert 200 removably attachable atop of the front surface 142. In use, the form insert 200 is mechanically secured as being surrounded by the upstanding sidewall 146 and two bosses 148A/B. In addition to the mechanical support with the upstanding sidewall 146 and two bosses 148A/B, the form insert 200 may be further provided with a peelable adhesive layer facing the front surface 142 to strengthen attaching force thereon. The upstanding sidewall 146 may provide mechanical support to secure the form insert 200 on the front surface 142 as well as a safety guard to prevent a tip of the dental tool from slipping off therefrom.

Further, the form insert 200 is a deformable material and serves a cleaning surface for wiping contaminated dental tools. The form insert 200 may contain disinfecting substances for additional cleaning power. The shape of the form insert 200 is generally flat and corresponds with the outer edge of the front surface 142. The form insert 200 may be disposable and replaceable with new one for each use. To facilitate the removal of the form insert 200, the compartment 150A/B of the device 100 may include at least one recess-lip 158 along the inner wall 156 for easy gripping of the form insert 200 as shown in FIGS. 1 and 3. The form insert 200 may comprise a polyurethane open cell form as a cleaning surface, a white poster board for sufficient rigidity, and Unifilm transfer tape therebetween to adhere them together. Further, the polyurethane open cell may have an approximate thickness of ¾ inch.

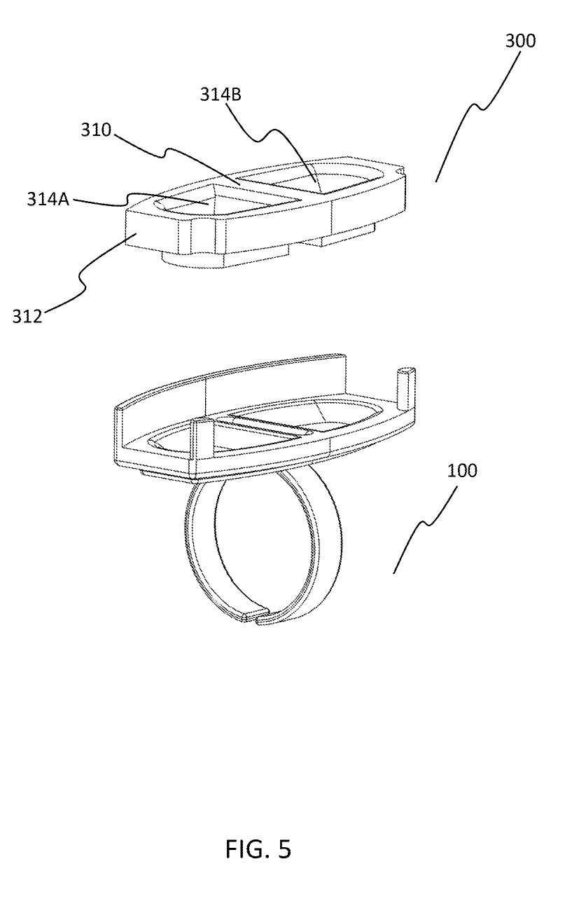

In yet another embodiment as shown in FIG. 5, the device 100 is further provided with a deep well insert 300 removably attachable atop of the front surface 142. In use, the deep well insert 300 is mechanically secured as being surrounded by said upstanding sidewall 146 and said bosses 148A/B. The deep well insert 300 comprises an overplay portion 310 positioned atop of the front surface 142, and a peripheral portion 312 downwardly extending from fringes of the overplay portion 310. The overplay portion 310 is shaped in accordance with the exterior geometry of the front surface 142 for creating a deeper well 314A/B than the compartment 150A/B in the platform 140. Thereby, the well 314A/B as provided can hold more volume of gel and alternately liquid without spillage.

The deep well insert 300 may comprise a single rigid layer of non-toxic compliant materials. Even chemical, chill, or heat sterilization may be applicable to the deep well insert 300 for reusability. Depending on the exterior shape of the platform 140, the deep well insert 300 may have more than one well 314A/B. Therein, multiple wells may be prefilled with various types of polishing pastes.

The method of using the device 100 may be achieved by performing the following steps: (a) providing at least one freehand ring 100; (b) placing the ring 110 on the finger so that the band 112 grips the finger and remain in place thereon; (c) adding gel or liquid type dental substances into the compartment 150A/B; (d) taking the substance therefrom using another hand not wearing the ring 110; and (e) sterilizing the freehand ring 100 to reuse next time.

The method of using the device 100 and the form insert 200 may be achieved by performing the following steps: (a) providing at least one freehand ring 100; (b) placing the ring 110 on the finger so that the band 112 grips the finger and remains in place thereon; (c) attaching the form insert 200 atop of the front surface 142; (d) wiping a contaminated dental tool on the from insert 200 as needed; (e) pinching the form insert 200 near the recess-lip 158 after use; and (f) removing the form insert 200.

The method of using the device 100 and the deep well insert 300 may be achieved by performing the following steps: (a) providing at least one freehand ring 100; (b) placing the ring 110 on the finger so that the band 112 grips the finger and remains in place thereon; (c) attaching the deep well insert 300 atop of the front surface 142; (d) adding gel or liquid dental substances into the well 314A/B; and (f) taking the substance therefrom using another hand not wearing the ring 110.

Although the foregoing examples describe some details of the present invention for purposes of clarity of understanding, the invention is not limited to the details provided. There are many alternative ways of implementing the invention. The disclosed examples are illustrative and not restrictive. Therefore, a numerous modifications or changes can be made to the invention but still within the scope of the invention as set forth in the claims.

1. A freehand ring for assisting a dental procedure comprising:
(a) a ring comprising a substantially circular band, a finger passage axis, a band top, and a pair of distal ends of said band, wherein said ends are positioned proximate each other and form a gap therebetween to be adjustable for fingers of various sizes; and
(b) a platform having a generally rectangular tray-like configuration, and comprising a front surface, a back surface opposed thereto, a long axis crossing a center of width, and a short axis crossing a center of length, wherein said front surface is further integral with an upstanding sidewall extending from a long periphery of said front surface, two upstanding bosses extending from two vertexes opposite of said upstanding sidewall, and at least one compartment defined by an opening at said front surface, a base recessed therefrom, and an inner wall extending upwardly from an outer edge of said base to form an interior space for receiving gel and alternately liquid; and
(c) wherein said band top is integrally connected at the center of said back surface, and said finger passage axis is generally parallel to said long axis and perpendicular to said short axis;

2. The device of claim 1, wherein said ring and said platform are formed as a monolithic structure.

3. The device of claim 2, wherein edges of said freehand ring are smoothed with general curves.

4. The device of claim 3, wherein said platform is provided with a prefilled polishing substance.

5. The device of claim 4, wherein said platform comprises two recessed compartments prefilled with different types of polishing substances and sealed with a peelable foil cover.

6. The device of claim 3, wherein said freehand ring comprises compliant resin materials selected from the group consisting of Dupont Zytel FG101L NC010, Dupont Zytel FG151L NC010, and Dupont Crastin FGS600F040 NC01.0.

7. The device of claim 6, wherein said freehand ring is adapted for sterilization for reusability.

8. The device of claim 1, wherein said platform is provided with a deformable form insert removably attached atop of said front surface, secured and surrounded by said upstanding sidewall and said bosses, and wherein a shape of said form insert is generally flat and corresponds with inner edges of said front surface.

9. The device of claim 8, wherein said compartment has at least one recess-lip around said inner wall for easy gripping of said form insert.

10. The device of claim 9, wherein said form insert is disposable.

11. The device of claim 10, wherein said form insert comprises a polyurethane open cell form as a cleaning surface, a white poster board for sufficient rigidity, and Unifilm transfer tape therebetween to adhere them together.

12. The device of claim 11, wherein said form insert is provided with a peelable adhesive layer facing said front surface for additional security to keep said form insert in place.

13. The device of claim 11, wherein said form insert is provided with a disinfecting substance for additional cleaning.

14. The device of claim 1, wherein said platform is provided with a deep well insert removably attached atop of said front surface, secured and surrounded by said upstanding sidewall and said bosses, wherein said deep well insert comprises an overplay portion positioned atop of said front surface, and a peripheral portion downwardly extending from fringes of said overplay portion, and wherein said overplay portion is shaped in accordance with the exterior geometry of said front surface for creating a deeper well than said compartment, whereby a well as provided can hold more volume of gel and alternately liquid without spillage.

15. The device of claim 14, wherein said deep well insert is a rigid single layer.

16. A method of using a freehand ring comprising:
(a) supplying at least one said freehand ring, each said freehand ring comprising:
a ring comprising a substantially circular band, a finger passage axis, a band top, and a pair of distal ends of said band, wherein said ends form a gap therebetween to accommodate fingers of various sizes;
a platform having a generally rectangular tray-like configuration; integrally comprising a front surface, a back surface opposed thereto, a long axis crossing a center of width, and a short axis crossing a center of length, wherein said front surface is further provided with an upstanding sidewall extending from a long periphery of said platform, two upstanding bosses extending from two corners facing said sidewall, and at least one compartment defined by an opening at said front surface, a base recessed therefrom, and an inner wall extending upwardly from an outer edge of said base to form an interior space for containing gel and alternately liquid;
wherein said band top is continually connected to the center of said back surface, and said finger passage axis is generally parallel to said long axis and perpendicular to said short axis;
a deformable form insert having a shape in accordance with inner edges of said front surface, whereby said form insert is removably attachable atop of said front surface as being secured and surrounded by said upstanding sidewall and said bosses; and
a deep well insert comprising an overplay portion shaped in accordance with the exterior geometry of said front surface for creating a deep well, and a peripheral portion downwardly extending from fringes of said overplay portion, whereby said deep well insert is removably attachable atop of said front surface as being secured and surrounded by said upstanding sidewall and said bosses; and
(b) placing said ring on a finger so that said band grips said finger and remains in place thereon;

17. The method of claim 16 further comprises adding gel and alternately liquid substance into said compartment, taking said gel and alternately said liquid therefrom using another hand not wearing said ring.

18. The method of claim 17 further comprises sterilizing said freehand ring for multiple uses.

19. The method of claim 16 further comprises attaching said form insert atop of said front surface, wiping a dental tool on said form insert as needed, pinching said form insert near said recess-lip after use, and removing said form insert.

20. The method of claim 16 further comprises attaching said deep well insert atop of said front surface, adding gel or liquid substance into said well, and taking said substance therefrom using another hand not wearing said ring.
